(19) United States
(12) Patent Application Publication
(10) Pub. No.: US 2011/0029062 A1
(43) Pub. Date: Feb. 3, 2011

(54) STENT USED IN BLOOD VESSEL

(76) Inventor: Sung-Gwon Kang, Gyunggi-do (KR)

Correspondence Address: BIRCH STEWART KOLASCH & BIRCH, PO BOX 747, FALLS CHURCH, VA, 22040-0747, US

(21) Appl. No.: 12/936,638

(22) PCT Filed: Jul. 22, 2008
(86) PCT No.: PCT/KR08/04263

§ 371 Date: Oct. 6, 2010

(30) Foreign Application Priority Data
Jul. 16, 2008 (KR) 10-2008-0068851

(51) Int. Cl. A61F 2/82

(52) U.S. Cl. 623/1.15

(57) ABSTRACT

The present invention discloses a stent for a blood vessel. The stent for a blood vessel comprises (i) an artificial blood vessel A made of fabric; (ii) an upper cylindrical stent B made of wires having a body B1 which is fixed by suture to the inner surface of said artificial blood vessel A and a upper portion T protruded out of the artificial blood vessel A in a form of a fallopian tube with the outer diameter gradually increasing toward the top end thereof; (iii) a middle and lower cylindrical stent C made of wire which is fixed by suture to the inner surface of said artificial blood vessel A and is made of a plurality of mutually separated repetitive units, wherein the unit structures of the repetitive units that make said middle and lower cylindrical stent C form rhombic cells.

### TECHNICAL FIELD

The present invention relates to a stent for a blood vessel, more specifically to a stent for a blood vessel which effectively prevents the phenomenon in which the blood passing through the blood vessel leaks out into the space between the front end portion and the rear end portion of the stent for a blood vessel that is installed on the blood vessel wall and in the blood vessel, and which is also easy to perform an operation therewith.

### BACKGROUND ART

The stent for a blood vessel consists of an artificial blood vessel (graft) made of fabric and a cylindrical stent made of wire and having a lengthwise axis, and is used to treat the phenomenon in which blood passing through the blood vessel leaks out of the blood vessel to the region around it.

As a stent for a blood vessel is widely used conventionally, there is one which consists of an artificial blood vessel (graft) made of fabric, and in which all the parts of the cylindrical stent made of wire and having a lengthwise axis are monolithically fixed by suture to the outer surface of the artificial blood vessel. In other words, such a stent for a blood vessel is a structure in which the whole of the cylindrical stent is inserted over the outside of the artificial blood vessel (graft).

But the conventional stent for a blood vessel described above has a problem in that a phenomenon, in which the blood passing through the blood vessel leaks out into the space between the front end portion and the rear end portion of the stent for a blood vessel that is installed on the blood vessel wall and in the blood vessel, occurs severely during an operation on the blood vessel.

As another stent for a blood vessel widely used conventionally, there is one which consists of an artificial blood vessel (graft) made of fabric and in which all the parts of the cylindrical stent made of wire and having a lengthwise axis are monolithically fixed by suture on the inner surface of the artificial blood vessel. In other words, such a stent for a blood vessel is a structure in which the whole of the cylindrical stent is inserted into the artificial blood vessel (graft) and fixed thereto.

But the conventional stents for a blood vessel described above have a problem in that operation is inconvenient because a phenomenon, in which the guide wire is caught on the cylindrical stent inserted into the artificial blood vessel, is severe during an operation of inserting the stent for a blood vessel into the blood vessel.

Yet another conventional stent is shown in FIG. 5, which is disclosed in Korean Patent Application No. 2008-33421. The conventional stent for a blood vessel comprises (i) an artificial blood vessel A made of fabric; and (ii) an upper cylindrical stent D1, a middle cylindrical stent D2 and a lower cylindrical stent D3 which are made of wire and separated from each other, wherein the upper cylindrical stent D1 and lower cylindrical stent D3 are monolithically fixed by suture on the inner surface of the artificial blood vessel A, and the middle cylindrical stent D2 is fixed by suture to the outer surface of the artificial blood vessel A, and the upper portion T of the upper cylindrical stent D1 has a shape of a fallopian tube with the diameter gradually increasing toward the front end thereof and is protruded out of the artificial blood vessel A, wherein the middle cylindrical stent D2 and the lower cylindrical stent D3 are made in a unit structure in which zigzag shape wires with a peak portion P and a valley portion V repeating alternately are repeated in a mutually separated condition, as shown in FIG. 6.

FIG. 5 is a photograph of a conventional stent for a blood vessel, and FIG. 6 is a partially enlarged view showing the unit structure of the middle cylindrical stent D2 and the lower cylindrical stent D3 that compose the stent for a blood vessel illustrated in FIG. 5.

The conventional stent for a blood vessel has an effect of effectively preventing a guide wire from making an operation difficult by being caught in the cylindrical stent during an operation of inserting the stent for a blood vessel into the blood vessel. But it has a problem that because the middle cylindrical stent D2 and the lower cylindrical stent D3 are composed of the aforementioned zigzag shape wires as shown in FIG. 6, self-expansibility is weak after it is inserted into the blood vessel, so it should be expanded by an external force using a ballooning apparatus, etc.

In the case of expanding by the external force the stent for a blood vessel inserted into the blood vessel as described above, there are problems such as: that an operation is complicated; that because the ballooning apparatus blocks blood circulation momentarily during operation, excessive load is applied to the heart of the patient or the blood vessel is damaged; and that when the ballooning apparatus is moved to the bottom of blood vessel by blood pressure, the stent for a blood vessel is also moved to the bottom of the blood vessel, so it is difficult to insert the stent for a blood vessel accurately into the desired position of the blood vessel.

### DISCLOSURE

### Technical Problem

Accordingly, it is an object of the present invention to provide a stent for a blood vessel which excels in self-expansibility, effectively prevents the phenomenon in which the blood passing through the blood vessel leaks out into the space between the front end portion and the rear end portion of the stent for a blood vessel attached to the blood vessel wall and in the blood vessel, and the operation process of expanding the stent for a blood vessel by external force using a ballooning apparatus can be omitted, so that safety of the patient is attained and it is easy to install in a desired position of the blood vessel.

### Technical Solution

In accordance with another aspect of the present invention, there is provided a stent for a blood vessel comprising: (i) an artificial blood vessel A made of fabric; (ii) an upper cylindrical stent B made of wires having a body B1 which is fixed by suture to the inner surface of said artificial blood vessel A and a upper portion T protruded out of the artificial blood vessel A in a form of a fallopian tube with the outer diameter gradually increasing toward the top end thereof; (iii) a middle and lower cylindrical stent C made of wire which is fixed by suture to the inner surface of said artificial blood vessel A and is made of a plurality of mutually separated repetitive units, wherein the unit structures of the repetitive units that make said middle and lower cylindrical stent C form rhombic cells. Preferably, each of the wires composing the upper portion T of the upper cylindrical stent B and the middle and lower cylindrical stent C is of a structure with two strands of wire twisted.

In the stent for a blood vessel, the wires composing the body B1 of the upper cylindrical stent B may be made of one strand of wire.

In the stent for a blood vessel, the body B1 of the upper cylindrical stent B may be composed of repetitive units having unit structures the wires form rhombic cells.

Preferably, the number of the rhombic cells in the repetitive unit of the body B1 of the upper cylindrical stent B formed circumferentially of the stent for a blood vessel is more than the number of the rhombic cells in the repetitive unit of the middle and lower cylindrical stent C formed circumferentially of the stent for a blood vessel.

Preferably, the number of the rhombic cells in the repetitive unit of the body B1 of the upper cylindrical stent B formed circumferentially of the stent for a blood vessel is 12 to 16.

Preferably, the number of the rhombic cells in the repetitive unit of the middle and lower cylindrical stent C formed circumferentially of the stent for a blood vessel is 5 to 10.

### Advantageous Effects

The present invention effectively prevents the blood passing through the blood vessel from leaking out into the space between the front end portion and the rear end portion of the stent for a blood vessel, and excels in self-expansibility, so that it is possible to omit the procedure of expanding, by external force using an apparatus such as a balloon, the stent for a blood vessel that is inserted into the human body during an operation of inserting the stent for a blood vessel into the blood vessel. Therefore, the operation is simplified, and it is possible to effectively prevent the phenomena such as burdening the heart of the patient because blood circulation is momentarily interrupted, damaging the blood vessel, or the stent for a blood vessel being moved down below the insertion position by blood pressure, which occur when the stent for a blood vessel is expanded by external force.

### DESCRIPTION OF DRAWINGS

These and other objects, features, aspects, and advantages of preferred embodiments of the present invention will be more fully described in the following detailed description, taken in conjunction with the accompanying drawings. In the drawings:

FIG. 1 is a photograph of a stent for a blood vessel according to the present invention;

FIG. 2 is a partially enlarged view showing a unit structure of the middle and lower cylindrical stent C and the upper portion T of the upper cylindrical stent that compose the stent for a blood vessel of the present invention;

FIG. 3 is a partially enlarged view showing a unit structure of the body B1 of the upper cylindrical stent B that composes the stent for a blood vessel of the present invention;

FIG. 4 is a perspective view of an artificial blood vessel A that composes the stent for a blood vessel of the present invention;

FIG. 5 is a photograph of an example of a conventional stent for a blood vessel; and

FIG. 6 is a partially enlarged view showing a unit structure of the middle cylindrical stent D2 and lower cylindrical stent D3 that compose the conventional stent for a blood vessel illustrated in FIG. 5.

### DESCRIPTION OF SYMBOLS OF THE MAJOR PARTS OF THE DRAWINGS

| | |
|---|---|
| a: artificial blood vessel (graft) | B: upper cylindrical |
| stent | |

B1: body of the upper cylindrical stent

| | |
|---|---|
| C: middle and lower cylindrical stent | S: supporting wire |

T: upper portion of the upper cylindrical stent

| | |
|---|---|
| V: wire valley portion | W1-W7: wire for composing stent |

### Best Mode

Below the present invention will be described in detail with reference to the accompanying drawings.

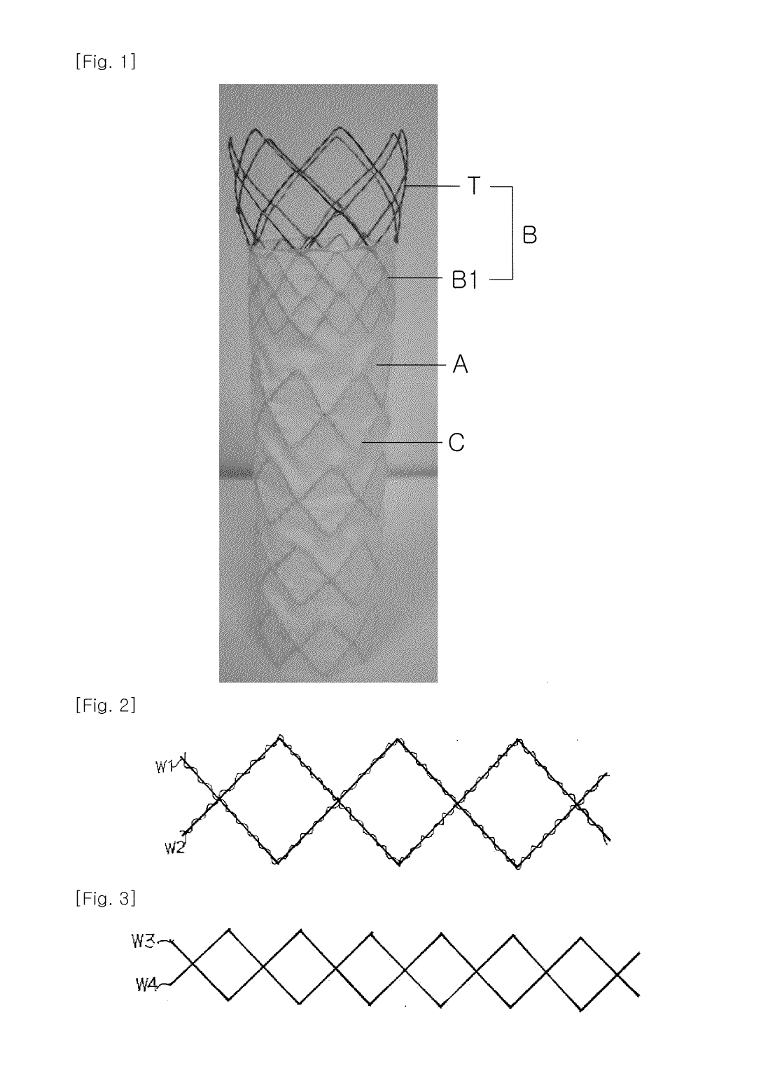

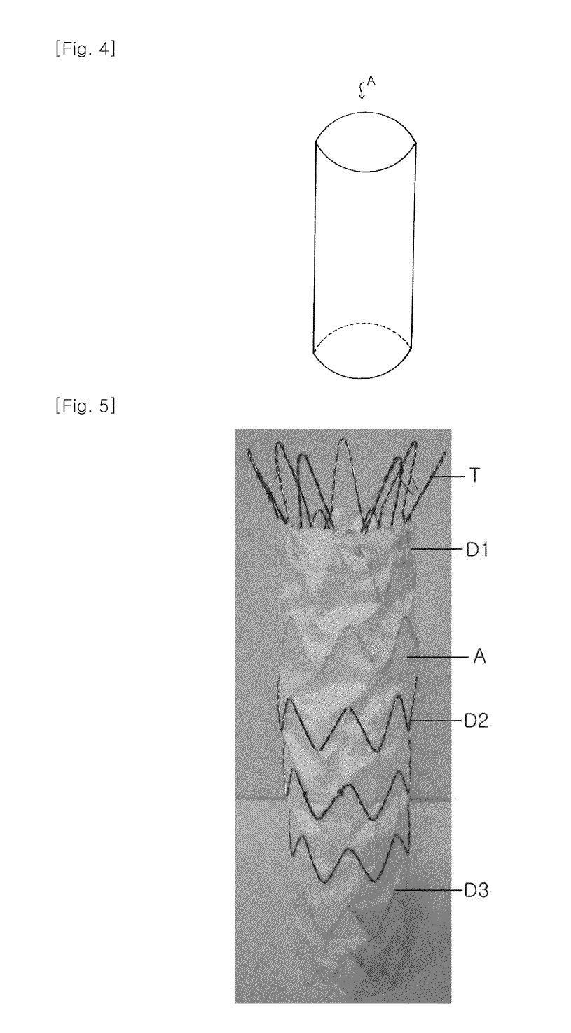

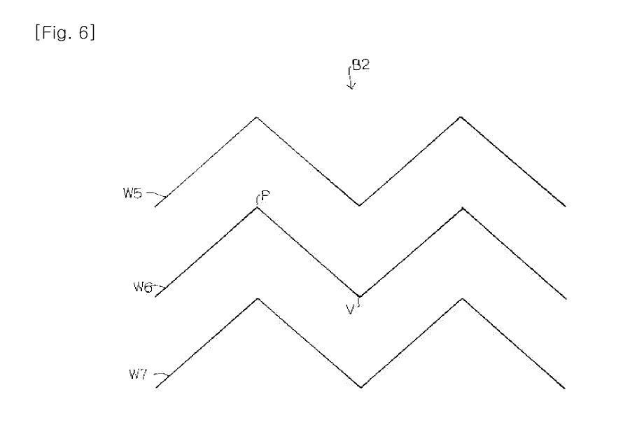

FIG. 1 is a photograph of a stent for a blood vessel according to the present invention.

In the stent for a blood vessel according to the present invention, an upper cylindrical stent B and a middle and lower cylindrical stent C are fixed by suture monolithically to the inner surface of an artificial blood vessel A.

And the upper portion T of the upper cylindrical stent B is protruded out of the artificial stent A in a form of a fallopian tube with the outer diameter gradually increasing toward the top end thereof.

As shown in FIG. 4, the artificial blood vessel A is a tubular type having a lengthwise axis and is made of fabric.

The artificial blood vessel A may have one or more, preferably one to five holes, punched for blood to pass therethrough.

The stent for a blood vessel of the present invention comprising the artificial blood vessel A with holes punched for blood to pass therethrough is useful for treating a branched blood vessel.

FIG. 4 is a perspective view of the artificial blood vessel A composing the stent for a blood vessel of the present invention.

Meanwhile, each of the upper portion T of the upper cylindrical stent B and the middle and lower cylindrical stent C are composed of repetitive units having a unit structure in which a wire W1 and a wire W2 form rhombic cells as shown in FIG. 2, and the wire W1 and wire W2 have a structure with two strands of wire twisted.

The above-mentioned repetitive units that make the upper portion T of the upper cylindrical stent B are mutually connected to be formed monolithically and the above-mentioned repetitive units that make the middle and lower cylindrical stent C are arrayed with a given interval maintained in a mutually separated condition lengthwise of the stent for a blood vessel.

FIG. 2 is a partially enlarged view illustrating the unit structure which makes the top and middle cylindrical stent C and the upper portion T of the upper cylindrical stent B that compose the stent for a blood vessel of the present invention.

Meanwhile, the body B1 of the upper cylindrical stent B is composed of repetitive units having unit structures in which a wire W3 and a wire W4 form rhombic cells as shown in FIG. 3, and each of the wire W3 and wire W4 is composed of one strand of wire.

FIG. 3 is a partially enlarged view showing the unit structure of the body B1 of the upper cylindrical stent B that composes the stent for a blood vessel of the present invention.

It is preferable for preventing blood from leaking into the space between the blood vessel and the stent for a blood vessel while maintaining self-expansibility, that the number of the rhombic cells in the repetitive unit of the body B1 of the upper cylindrical stent B formed circumferentially of the stent for a blood vessel is more than the number of the rhombic cells in the repetitive unit of the middle and lower cylindrical stent C formed circumferentially of the stent for a blood vessel.

Specifically, it is more preferable that the number of the rhombic cells in the repetitive unit of the body B1 of the upper cylindrical stent B formed circumferentially of the stent for a blood vessel is 12 to 16 and the number of the rhombic cells of the repetitive unit of the middle and lower cylindrical stent C formed circumferentially of the stent for a blood vessel is 5 to 10.

It is more preferable for fixing the stent for a blood vessel on the blood vessel wall that fixing hooks are formed at the upper portion T of the upper cylindrical stent.

As mentioned above, in the stent for a blood vessel according to the present invention, the middle and lower cylindrical stent C forms rhombic cells with the wire W1 and the wire W2 crossing each other, and furthermore the wire W1 and wire W2 are of a structure with two strands of wire twisted, so self-expansibility is excellent. Therefore, it is possible to omit the procedure for expanding by external force the stent for a blood vessel inserted into the blood vessel using a ballooning apparatus, etc.

As a result, it has advantages that operation is simplified, that it is possible to effectively prevent burdening the heart of the patient during an operation or damaging the blood vessel, and that the stent for a blood vessel can be located accurately on the desired position in the blood vessel.

In addition, because the stent for a blood vessel according to the present invention is of such a structure in which the body B1 of the upper cylindrical stent B forms more rhombic cells than those in the middle and lower cylindrical stent C circumferentially of the stent for a blood vessel as the wire W1 and the wire W4 cross each other, it has an advantage that it more effectively prevents blood from leaking into the space between a blood vessel and the stent for a blood vessel.

### INDUSTRIAL APPLICABILITY

The present invention effectively prevents the blood passing through the blood vessel from leaking out into the space between the front end portion and the rear end portion of the stent for a blood vessel, and excels in self-expansibility, so that it is possible to omit the procedure of expanding, by external force using an apparatus such as a balloon, the stent for a blood vessel that is inserted into the human body during an operation of inserting the stent for a blood vessel into the blood vessel.

Although the present invention has been described in connection with the exemplary embodiments illustrated in the drawings, it is only illustrative. It will be understood by those skilled in the art that various modifications and equivalents can be made to the present invention. Therefore, the true technical scope of the present invention should be defined by the appended claims.

1. A stent for a blood vessel comprising:
(i) an artificial blood vessel A made of fabric;
(ii) an upper cylindrical stent B made of wires having a body B1 which is fixed by suture to the inner surface of said artificial blood vessel A and a upper portion T protruded out of the artificial blood vessel A in a form of a fallopian tube with the outer diameter gradually increasing toward the top end thereof;
(iii) a middle and lower cylindrical stent C made of wire which is fixed by suture to the inner surface of said artificial blood vessel A and is made of a plurality of mutually separated repetitive units,
wherein the unit structures of the repetitive units that make said middle and lower cylindrical stent C form rhombic cells.

2. The stent for a blood vessel of claim 1, wherein each of the wires composing the upper portion T of the upper cylindrical stent B and the middle and lower cylindrical stent C is of a structure with two strands of wire twisted.

3. The stent for a blood vessel of claim 1, wherein the wires composing the body B1 of the upper cylindrical stent B are made of one strand of wire.

4. The stent for a blood vessel of claim 1, wherein the body B1 of the upper cylindrical stent B is composed of repetitive units having unit structures the wires form rhombic cells.

5. The stent for a blood vessel of claim 1, wherein the number of the rhombic cells in the repetitive unit of the body B1 of the upper cylindrical stent B formed circumferentially of the stent for a blood vessel is more than the number of the rhombic cells in the repetitive unit of the middle and lower cylindrical stent C formed circumferentially of the stent for a blood vessel.

6. The stent for a blood vessel of claim 5, wherein the number of the rhombic cells in the repetitive unit of the body B1 of the upper cylindrical stent B formed circumferentially of the stent for a blood vessel is 12 to 16.

7. The stent for a blood vessel of claim 5, wherein the number of the rhombic cells in the repetitive unit of the middle and lower cylindrical stent C formed circumferentially of the stent for a blood vessel is 5 to 10.

8. The stent for a blood vessel of claim 4, wherein the number of the rhombic cells in the repetitive unit of the body B1 of the upper cylindrical stent B formed circumferentially of the stent for a blood vessel is more than the number of the rhombic cells in the repetitive unit of the middle and lower cylindrical stent C formed circumferentially of the stent for a blood vessel.

9. The stent for a blood vessel of claim 8, wherein the number of the rhombic cells in the repetitive unit of the body B1 of the upper cylindrical stent B formed circumferentially of the stent for a blood vessel is 12 to 16.

10. The stent for a blood vessel of claim 8, wherein the number of the rhombic cells in the repetitive unit of the middle and lower cylindrical stent C formed circumferentially of the stent for a blood vessel is 5 to 10.
